(19) United States
(12) Patent Application Publication
(10) Pub. No.: US 2006/0032823 A1
(43) Pub. Date: Feb. 16, 2006

(54) Disinfection of reverse osmosis membrane

(76) Inventors: Alan D. Harrison, Douglasville, GA (US); Patrick Sisk, Marietta, GA (US)

Correspondence Address: SMITH, GAMBRELL & RUSSELL, LLP, 1230 PEACHTREE STREET, N.E., SUITE 3100, PROMENADE II, ATLANTA, GA, 30309-3592, US

(21) Appl. No.: 10/526,728

(22) PCT Filed: Sep. 4, 2003
(86) PCT No.: PCT/US03/27768

§ 371 Date: Mar. 4, 2005

Related U.S. Application Data
(60) Provisional application No. 60/408,095, filed on Sep. 4, 2002.

(51) Int. Cl. C02F 1/76

(52) U.S. Cl. 210/754

(57) ABSTRACT

A process for treating a reverse osmosis membrane with a biocide to kill bacteria on or in the vicinity of a membrane by contacting a reverse osmosis membrane with a halogen biocide in combined form which slowly releases halogen in a sufficient amount to disinfect the membrane and to kill any bacteria and thereby eliminate or prevent forming a biofilm on said membrane.

### REFERENCE TO A RELATED APPLICATION

The present application claims the benefit of our co-pending provisional application 60/408,095 filed Sep. 4, 2002, which is relied on and incorporated herein by reference.

### BACKGROUND OF THE INVENTION

Throughout the world, clean water both for drinking and industrial uses is in short supply. Because of this, the use of “desalination” technologies is becoming wide spread. The two major technologies used for desalination are thermal desalination and reverse osmosis. Thermal desalination involves the use of heat to turn water into steam, which is then condensed and used. The production of steam leaves behind most impurities so the water condensed from the steam is very clean and pure. The other technology, reverse osmosis, involves the use of a selectively permeable membrane, which will allow the passage of water, but not allow the passage of salts and other undesirable contaminants. By applying pressure to water against this membrane, the water is forced through the membrane and the contaminants, such as salts, are left behind. Reverse osmosis uses two types of selectively permeable membranes, acetate and polyamide. The acetate membranes are considered to be “old” technology and are undesirable because of low efficiency, which leads to high cost of water produced. The new technology, polyamide membranes, leads to much greater efficiency, which makes water produced from reverse osmosis much more economically feasible. Virtually all new reverse osmosis (RO) construction is done using polyamide membranes.

To maximize the membrane surface area in the smallest possible volume, polyamide membranes are typically spiral wound. Water is forced into the small spaces between the layers, through the membranes and pure water comes out the other end of the membrane housing. Because the spaces between the layers are so small, any material that collects in these spaces interferes with the functioning of the membrane by slowing down the flow, or increasing the pressure needed to move the water through the unit. Typical “matter” that can clog these units is silt from incoming water, inorganic scale which is often formed or precipitated in the presence of pressure changes or changes in concentration and biofouling. Silt is relatively easily removed by the appropriate use of pre-filters and inorganic scale is currently prevented by the addition of chemical additives or the use of acid cleaning when inorganic scale does form. Inorganic scale can be formed by calcium, barium, magnesium, sodium, strontium, chloride, sulfide, silicates, phosphate, bicarbonate, carbonate and sulfate.

Thus, the problem confronting the industry is the formation of biofouling or “biofilms” in the spaces between the layers of the membrane. Biofouling is a common problem in RO because of the source waters used. Typical sources of water for RO are brackish surface waters (inlets, bays, streams, etc.) and seawater. These water sources are typically home to a large population of bacteria. When bacteria are moved into small spaces against membranes, they tend to colonize the surface and form a thick mat called a biofilm. Other sources of biofouling include humic acid, algae and fungi. This is the genesis of biofouling in the RO systems.

Reverse osmosis membranes are susceptible to contamination by waterborne micro-organisms, which ultimately leads to fouling of the membrane surface and results in reduced membrane performance.

Biofilm organisms may irreversibly damage the membrane by degrading the membrane polymer itself.

Microorganisms attached to the membrane surface can also act as nucleation sites for scale and other deposit formation. Removal of biofilm is therefore necessary to ensure optimal membrane efficiency.

Currently several techniques are used for handling biofouling in reverse osmosis systems. One common approach is to just ignore biofouling and replace membranes that become too fouled. This is not an attractive approach because biofouling can significantly shorten the life of a membrane, which shortens the interval between membrane replacement, thereby increasing the cost of the water produced.

Another approach is the use of non-oxidizing biocides, such as DBNPA (dibromonitrilopropionamide), these compounds are usually very effective at killing and removing the bacteria, but are not approved for use in drinking water and may be toxic. What this means is that any water containing these non-oxidizing biocides cannot be used as drinking water. Therefore, when using a non-oxidizing biocide to remove bacteria from a membrane, water produced must be discarded to prevent anyone from drinking the water containing the non-oxidizing biocide. This represents a waste of produced water and has an associated monetary cost. It also means that during the treatment period, no water is being produced for consumption.

Some commonly used techniques for controlling biological fouling in RO systems are listed below:
1) Chlorine (i.e. Sodium Hypochlorite;Chlorine Gas) with subsequent dehalogenation (with a reducing agent such as Sodium Metabisulfite or Sodium Bisulfite) to prevent active halogen from contacting the membrane surface
2) Peracetic Acid
3) Dibromonitrilopropionamide (DBNPA)
4) Ultraviolet Light (UV)
5) Ozone

Chlorine is approved for use in the treatment of drinking water and is very effective at removing and killing bacteria. However, while acetate membranes are compatible with chlorine, polyamide membranes are not. It is well known that chlorine quickly degrades polyamide membranes. Most manufacturers of polyamide membranes give explicit warnings against exposing their membranes to chlorine for more than 1000-ppm hours. This is not nearly enough exposure time to effectively keep a membrane clean and to extend the life of the membrane by the use of chlorine. Free halogens, in general (chlorine, bromine and iodine) are all known to cause this membrane breakdown. Some RO plants still use chlorine, a free halogen, but they have to add a process for the removal of chlorine before the water gets to the membrane. This added step is inconvenient and adds cost.

Accordingly, it is an object of the present invention to disinfect reverse osmosis membranes in a technically effective and efficient manner.

### SUMMARY OF THE INVENTION

The above and other objects of the present invention can be achieved by treating the reverse osmosis membranes with 1) an oxidizing biocide substance that contains a halogen in the +1 oxidation state, and 2) a nitrogen containing compound which contains at least one nitrogen atom in the imide or amide form, such that the halogen loosely binds with the nitrogen thereby forming combined halogen. This can be achieved by treating with compounds with 1 and 2 separately such that both materials are present at the membrane surface at the same time, or by treating with a compound, which contains both 1 and 2 in the same compound. Some examples of 1 (materials containing halogen in the +1 oxidation state) are sodium hypochlorite, activated sodium bromide, chlorine gas, elemental bromine, bromine chloride, or calcium hypochlorite. Some examples of 2, (materials containing at least one nitrogen atom in the imide or amide form), are dimethylhydantoin, ammonia, benzene sulfamide, sulfamic acid, and cyanuric acid. Some examples of compounds, which contain both 1 and 2 above in the same compound, are bromochlorodimethylhydantoin, trichloroisocyanuric acid, bromosulfamate, and dichloromethylethylhydantoin. The preferred compound for purposes of this invention is bromochlorodimethylhydantoin (BCDMH).

### BRIEF DESCRIPTION OF DRAWINGS

The present invention will be further understood with reference to the accompanying drawings, wherein:

FIG. 1-1 is a graph showing tensile strengths vs. exposure time for treated polyamide fibers;

FIG. 1-2 is a graph showing % elongation vs. exposure time for treated polyamide fibers;

FIG. 1-3 is a graph showing Young's modulus vs. exposure time for treated polyamide fibers;

FIG. 2-1 is a schematic flow diagram of a flat plate test cell used to test the present invention;

FIG. 2-2 is a graph showing a plot of BCDMH content and permeate flux;

FIG. 2-3 is a graph showing the effect on permeate flux as a function of sodium hypochlorite content;

FIG. 3-1 is a schematic flow diagram of the St. Croix, Virgin Islands reverse osmosis plant for carrying out the process of the present invention;

FIG. 3-1 is a graph showing the permeate concentration versus time with BCDMH added;

FIG. 3-3 is a graph showing salt rejection percentage as a function of time;

FIG. 4-1 is a schematic flow diagram of the reverse osmosis plant at another location;

FIG. 4-2 is a graph showing change in permeate flow rate with time; and

FIG. 4-3 is a graph showing the change in percent salt rejection with time.

### DETAILED DESCRIPTION OF INVENTION

In carrying out the present invention, the oxidizing halogen-containing biocide substance is fed into the stream of seawater upstream from the reverse osmosis membrane to provide 0.05 to 4 ppm total halogen, preferably 0.1 to 2 ppm total halogen, and most preferably, 0.5 to 1 ppm total halogen, in the vicinity of or in contact with said membrane.

The biocide can be in the form of a solid such as a compact; e.g. tablets or granules, which is then dissolved and the concentrated solution is fed into the seawater stream for desalination treatment. A suspension of the biocide can also be used in which case the suspension is fed directly into the seawater stream without dilution as it is a pumpable liquid.

Typically the process is carried out at ambient temperature and standard operating parameters of pressure.

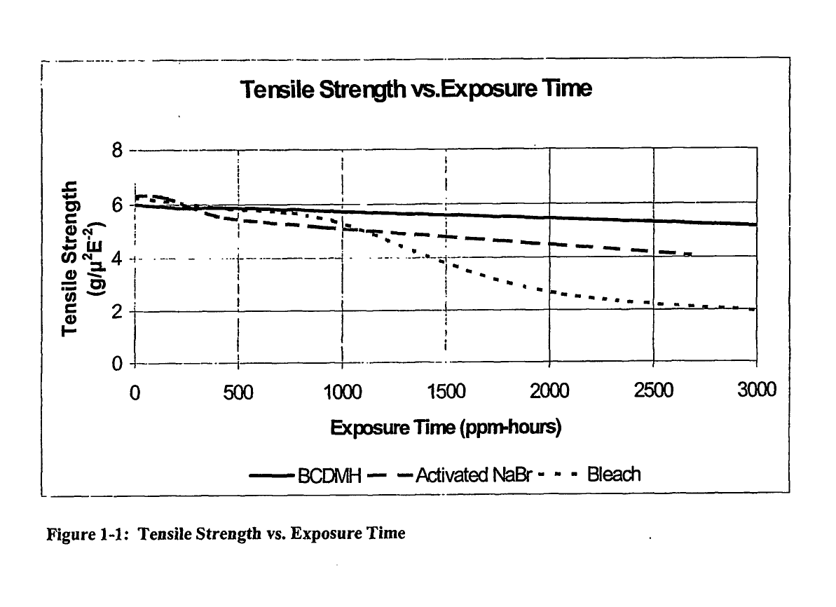

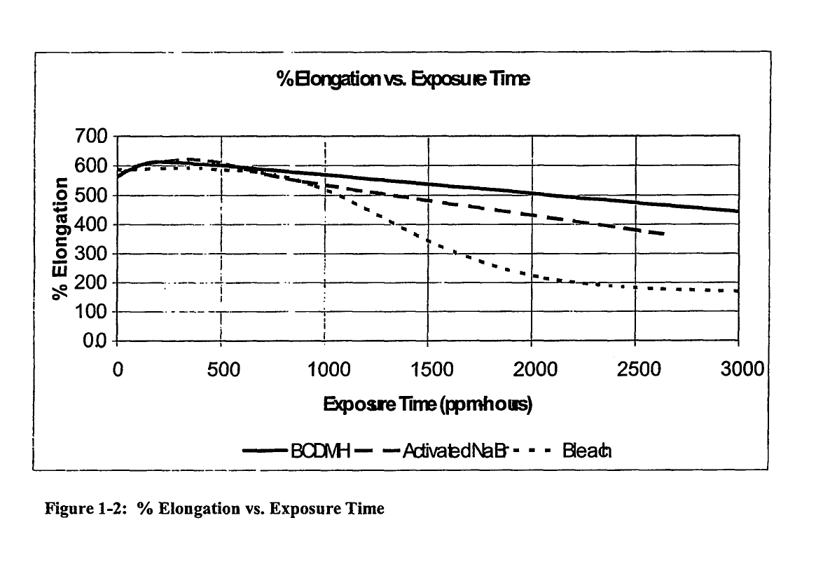

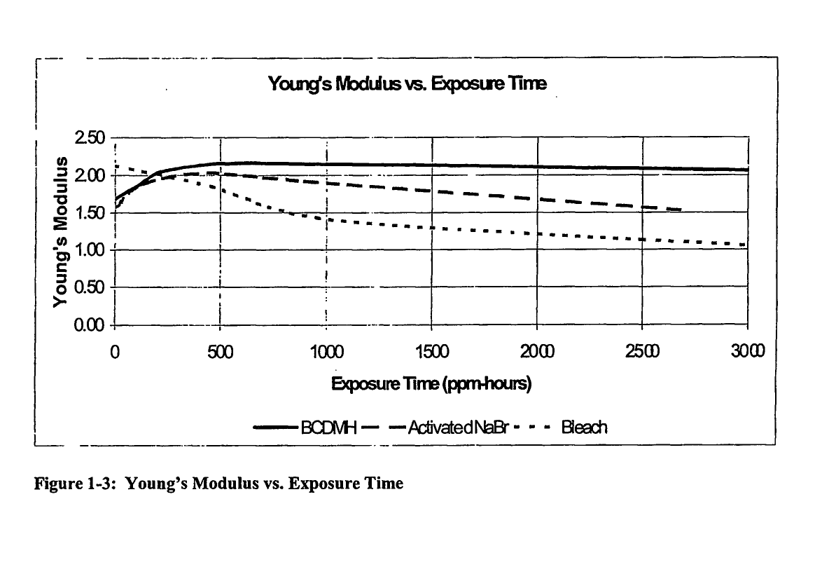

BCDMH (an oxidizing biocide which kills with chlorine and bromine) can be used without significantly shortening the life of the membrane. Experiments have been performed exposing raw polyamide fibers to BCDMH, chlorine and bromine. (See Example 1 and FIGS. 1-1 to 1-3.) These experiments show that the physical characteristics of these fibers, (tensile strength, elongation and Young's modulus) are not negatively effected by BCDMH to the same degree or as quickly as they are by chlorine or bromine. Much of this information has been published, but only as it relates to the use of polyamide materials in the paper industry, with respect to halogen in uncombined form. No information has been released on polyamide materials or structures related to RO uses.

While not wishing to be bound to any theory, it is believed that this effect is caused by the presence of DMH in BCDMH. The DMH (dimethylhydantoin) is an organic “backbone” that serves to carry the bromine and chlorine by loosely binding it with the imide and amide nitrogen atoms in the DMH molecule. In effect what this does is make most of the bromine and chlorine unavailable for reactions with the membranes. As bromine and chlorine are released they are only available at very low concentrations. This effect has been seen in other water treatment applications. The presence of mostly “combined” halogen (that which is bound to the nitrogen) is known to reduce unwanted effects such as corrosion or interaction with other desirable chemicals in the water without reducing the biocidal effect.

Clearly, this invention applies to the use of BCDMH to disinfect RO membranes. However, since the invention is related to prevalence of “combined” vs. “free” halogen, it is believed that any material that will cause halogen to be present mostly in the combined form could be used. This would include any materials that include nitrogen atoms in the imide or amide form. Two examples are sulfamic acid and benzene sulfonamide, two materials that are currently used in industry to combine with halogens and slowly release. So, for example, bromosulfamate could be manufactured and sold and should be within the scope of this invention.

Another possible approach would be the separate addition of halogen and a material containing an imide or amide nitrogen that would combine with the halogen and cause it to be present predominantly in the combined form. Two examples of this would be the separate addition of DMH and bromine and the separate addition of sulfamic acid and chlorine.

The current invention would allow the use of oxidizing biocides for disinfection of RO membranes. Currently there is no known way to use oxidizing biocides on RO membranes without significantly shortening the life of the membrane or adding the labor or cost intensive step of removing the halogen from the water before it contacts the membrane. This would also allow the use of biocides, which are approved for use in treating drinking water. This will eliminate the need for discarding water during the biocidal treatment as is currently done with non-oxidizing biocides.

Several methods were used during this investigation to support compatibility of bromine compounds in Polyamide Reverse Osmosis (RO) membranes.
Exposure of polyamide fibers to various combined and uncombined halogens
Flat plate test cell-laboratory test to study a small surface of the membrane
Field test at St. Croix
RO pilot plant unit

### EXAMPLE 1

### Compatibility Testing of Polyamide Fibers with Various Forms of Halogen

For this work polyamide fibers were chosen for testing instead of directly using polyamide membrane material. The materials are chemically identical, and the use of fibers allows for easy analysis of physical parameters such as tensile strength to determine if any chemical attack has occurred on the polyamide material.

Samples of TM5000 polyamide fibers were prepared to simulate long term treatment with BCDMH, activated sodium bromide (using bleach, sodium hypochlorite, as activator), and bleach (sodium hypochlorite). Treatment time in ppm-hours total halogen as Cl₂ was calculated for several different concentrations. These samples were evaluated for tensile strength, % elongation, and Young's Modulus.

Treatment duration is measured in ppm-hours. By preparing a treatment curve showing the concentration of the total halogen as chlorine vs. time of use, the amount of halogen exposed to the felt material can be expressed in ppm-hours by integrating the treatment curve over a specific time interval. In other words, 1 ppm-hour equals 1 hour of exposure to 1 ppm of halogen (expressed) as chlorine. 10 hours of exposure to 0.5 ppm would equal 5.0 ppm-hours. Finally, 150 hours of exposure to 0.2-ppm halogen as chlorine would equal 30.0 ppm-hours. Simply stated, ppm-hours is calculated by multiplying ppm halogen by exposure time.

Polyamide fibers were treated with different concentration levels of halogen and were allowed to maintain contact for about 48 hours. The pH of the medium was maintained at 6.8 via phosphate buffer solution. During this time, the total halogen level was determined at 2, 5, 24, and 48 hours. Resulting treatment curves were constructed from the data. The integrals (ppm-hours) were also determined for this data. After the contact period, the polyamide fiber samples were washed, neutralized with a dilute thiosulfate solution, rinsed with deionized water, and allowed to dry.

Samples from all three treatment processes were analyzed for tensile properties.

### Methods and Results for Polyamide Compatibility Testing

The polyamide fibers were examined by several methods to evaluate the durability of the material. These methods included stress/strain and cross sectional area studies. Samples from BCDMH, activated sodium bromide (NaBr), and bleach (sodium hypochlorite) treated fibers were analyzed and data was collated.

Cross Sectional Area. Diameter measurements were carried out using the Fiber Dimensional Analysis System (FDAS), which incorporates the Mitituyo laser scanner (Dia-Stron Limited, Andover, UK). The fiber cross-sectional area was measured with a laser-scanning micrometer supplied by Diastron Ltd., UK (Mitutoyu, Model LS3100). A fiber sample was placed between a 1.0 mW 670-nm laser and a detector. The sample was slowly rotated. The detector analyzed different shadows caused by the rotating fiber. Data obtained was used to determine major and minor axes from the fiber and resultant cross sectional areas were determined assuming an elliptical cross-section (9). Tabulated data for the fiber cross sectional areas are found in Table I.

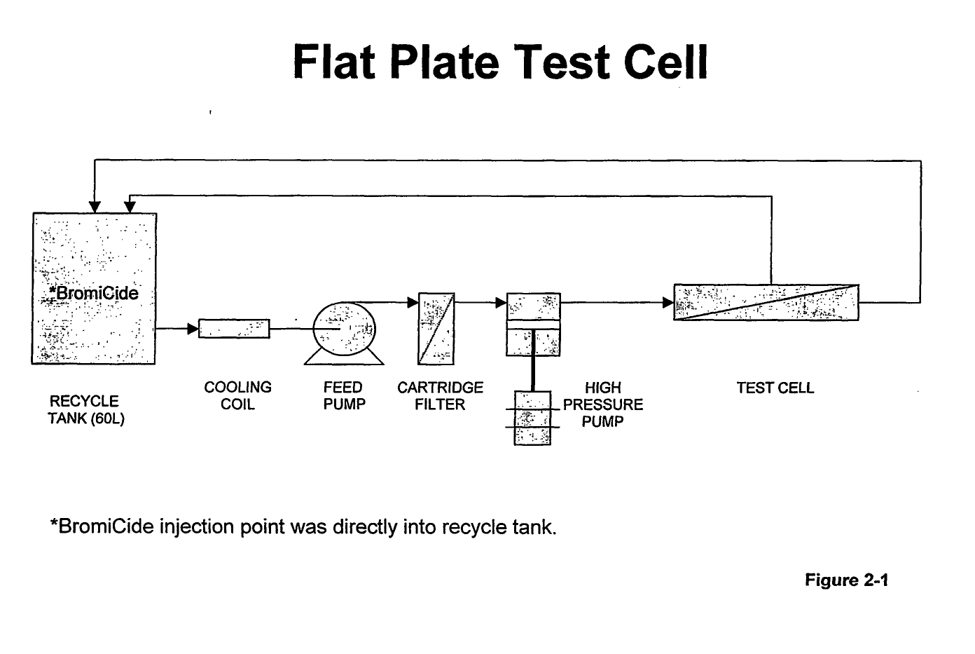

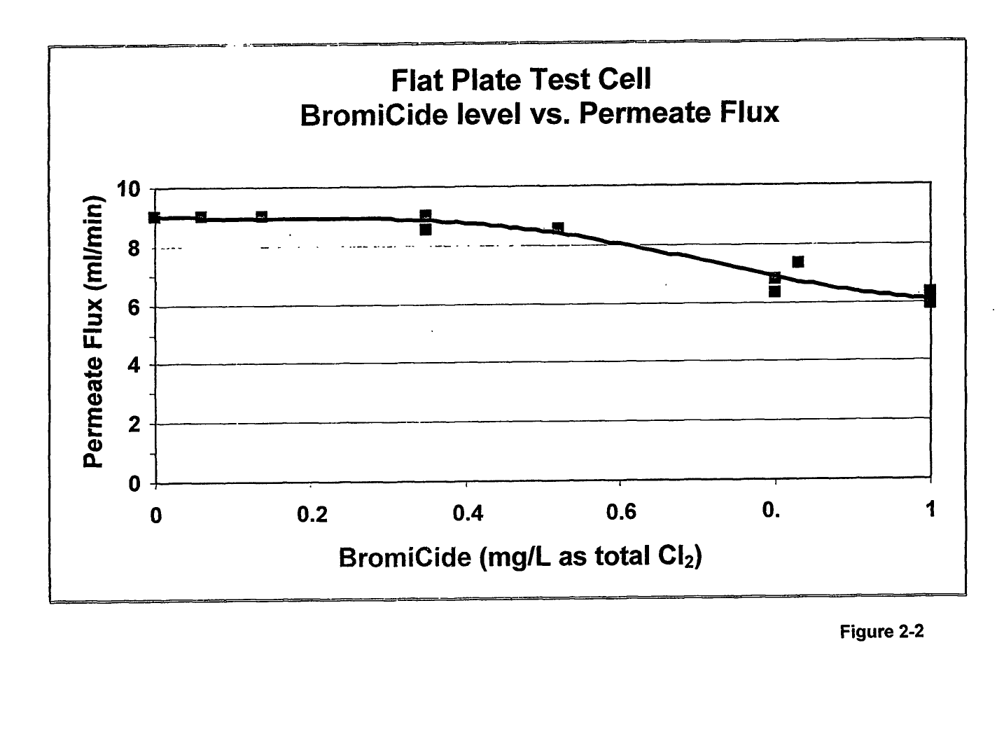

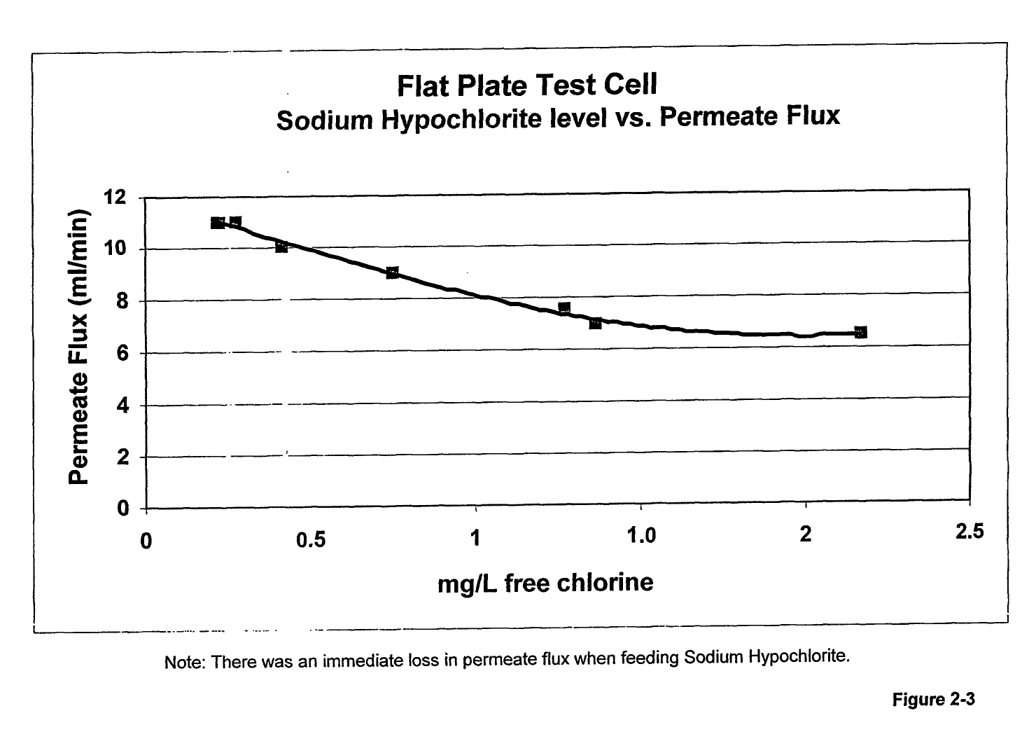

Stress/strain Evaluation. Stress and strain studies were carried out using the MTT600 Automated Tensile Tester (MTT600 Autosampler with a 100 fiber cassette, Dia-Stron Limited, Andover, UK). The samples were transferred to the tensile tester after making cross-sectional area measurements. Stress measurements were made and collected real-time while monitoring % fiber elongation. Three pieces of data were extracted or calculated from each test: % elongation at break point, tensile strength, and Young's Modulus. In order to allow for interpretation of the exposure differences vs. fiber attack, the individual results were plotted against total exposure time in ppm-hours and are shown in FIG. 1-1, FIG. 1-2, and FIG. 1-3. The graphs show that the fibers treated with sodium hypochlorite exhibited a significant loss in tensile properties compared to those treated with BCDMH. The activated sodium bromide treated samples were also affected but not as severely as the bleach treated samples. The tensile strength and % elongation dropped off significantly after about 1000 ppm-hours of treatment for bleach treated samples and the activated sodium bromide treated samples.

### Discussion of Polyamide Compatibility Results

Cross Sectional Area. The cross sectional area is an important piece of information regarding the tensile properties of fibers. The cross sectional areas of these fibers were analyzed using a laser to determine the range of the diameter of a fiber as it is rotated 180 degrees. This data was used to model the cross sectional area with minimum and maximum diameter of an ellipse. The cross sectional areas of the fibers were calculated using the formula for determining the area of an ellipse.

The cross sectional areas for all of the fibers examined showed that very little change in area was observed. This indicated that the fibers did not undergo any physical size change in the radial direction. That is, no swelling or shrinkage of the fibers was observed.

Tensile Strength. The tensile strength of the material is obtained by determining the amount of force required to break a fiber cross sectional mass (10). Test specimens were prepared and analyzed using a miniature tester with an autosampler, MTT 600 series, from Diastron Ltd., UK. The fibers were crimped inside a special cell that allowed a specific weigh to be placed along the length of the fibers while the specific dimensions of the fiber were measured. Fifty fibers from each sample were examined. The data shows that the samples treated with BCDMH have a higher tensile strength than the samples treated with sodium hypochlorite or activated sodium bromide. The approximate ppm-hours for a paper machine felt exposed to water treated with a halogenated biocide for at a 0.5 ppm residual, would be about 1792 ppm-hours halogen (as chlorine) for a 5 month period. The data shows that after 5 months; the chlorine based biocide has reduced the tensile strength of the fibers to about 50% of its original value, the activated sodium bromide biocide has reduced the tensile strength of the fibers to about 75% of its original value, and the BCDMH treated fibers have maintained more than 90% of their original tensile strength.

% Elongation. Elongation reflects how far a fiber can be extended before failure occurs. Elongation is an important factor in determining general fiber strength as it describes the fiber's ability to return to its original shape after undergoing stress. The data shows that the elongation of the fibers tested with sodium hypochlorite decreased substantially from about 58% to about 27% for a half year time span assuming a 0.5 ppm continuous residual (see Tensile Strength section above). The fibers treated with activated sodium bromide decreased from about 56% to about 45%. The fibers treated with BCDMH decreased from about 55% to about 52% for the same simulated treatment span. The large drop in elongation for the sodium hypochlorite treated fibers indicates the fibers become more brittle with time. The loss in the elongation for the activated sodium bromide samples indicates a somewhat less brittle condition. Both of these conditions indicate chemical breakdown of the polyamide fiber.

Young's Modulus. The Young's Modulus is related to the elasticity of fiber material. If you plot the stress on a fiber against the resulting % elongation, the slope of the curve formed before the yield phase is the modulus of the fiber. The fiber samples treated with sodium hypochlorite showed a decrease in tensile strength, elongation, and modulus. This indicates that over time the samples tend to become more rubbery with a decrease in tensile strength. The samples treated with activated sodium bromide also indicate a tendency to become rubbery over time although not as much as the fibers treated with sodium hypochlorite. The samples treated with BCDMH do not decrease in tensile strength and elongation nearly as much as sodium hypochlorite or activated NaBr. The modulus decreased very little. This indicates that the polyamide material undergoes vastly reduced chemical attack by the BCDMH as compared to the activated sodium bromide or the sodium hypochlorite.

### Introduction for Examples 2-4

Examples 2-4 utilize different types of membrane systems to evaluate exposure to various halogens and their associated concentrations. Key performance parameters such as permeate flux, normalized permeate flow rate and percentage salt rejection are indicators for determining membrane system(s) performance. Permeate flow rate, often referenced as permeate flux, is measured in flow volume per unit membrane surface area per unit time. Percentage salt rejection refers to the amount of solids that a particular membrane is capable of removing. Normalized permeate flow rate factors in a temperature correction coefficient, which is based upon a standard temperature of 25° C. This correction factor accounts for temperature variances that may occur with seasonal or feedwater changes. When new, polyamide membranes, such as those utilized in these three examples, are initially evaluated to create a baseline performance curve. Establishing a baseline is very important since future operating parameters are compared with this initial baseline data. This is referred to as normalization. Normalized data, used for monitoring performance, provides an indication of system declines. A 10% normalized permeate flow rate or salt rejection decline and/or a rise in differential pressure of 20% indicate membrane fouling and the need for an offline cleaning program or membrane change out.

### EXAMPLE 2

### Flat Plate Test Cell (FPTC)

This equipment is designed to test a small section of RO membrane under realistic pressures. (FIG. 2-1 provides a schematic of the flat cell apparatus.)

The unit has typically been used for:
Approvals for additive/membrane compatibility

| | |
|---|---|
| Evaluations for: | Scale control agents |
| | Biological control agents |

Conditions of operation

| | |
|---|---|
| Membrane type: | Thin film composite (HR98PP) |
| | (Danish Separations Systems AS) |
| Membrane Size: | 20 × 20 cm (as ordered) |
| | 15 × 20 cm (used on FPTC) |
| Parameters | Permeate flow rates |
| monitored: | Total Halogen |
| | Permeate conductivity |
| Water type: | Sodium chloride brine: TDS 35,000 mg/L |
| Products tested: | Bromochlorodimethylhydantoin (BCDMH); |
| | BromiCide ® from BioLab, Inc. |
| | Sodium Hypochlorite (Bleach) |
| Method used to | To monitor the amount of halogen, a DPD (N, |
| measure halogen: | N-diethyl-p-phenylenediamine) test kit was |
| | used. |
| Halogen dose | 0-1.0 mg/L (total chlorine) 0-2.5 mg/L (free halogen) |
| range: | |

Findings/Conclusion

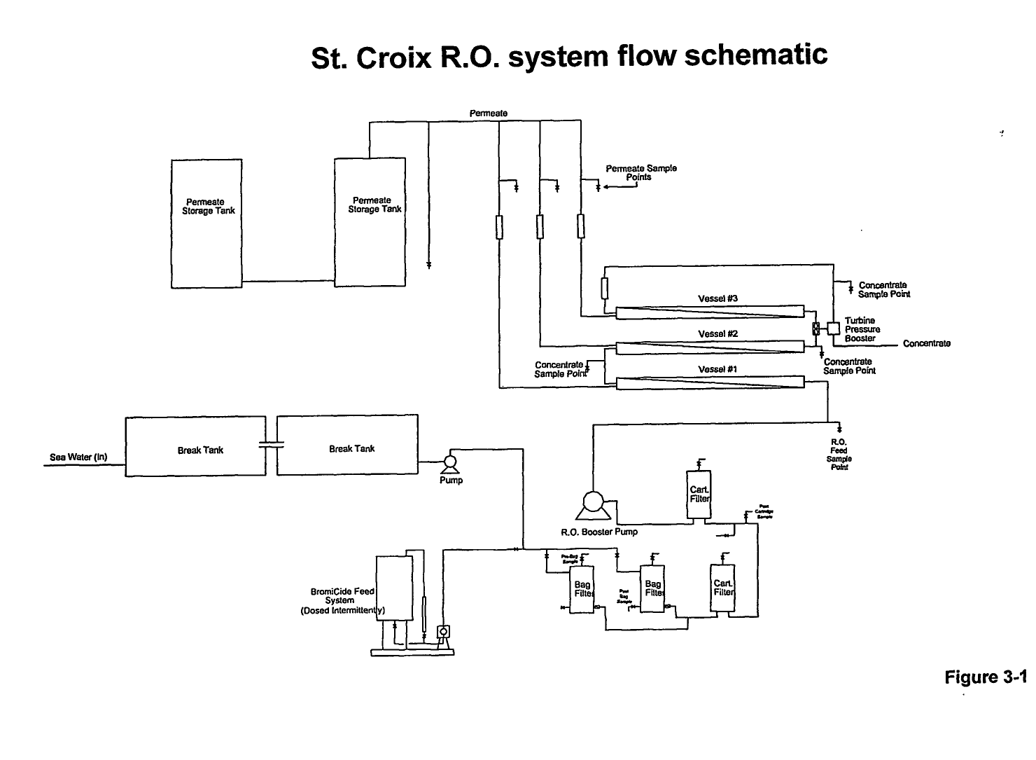

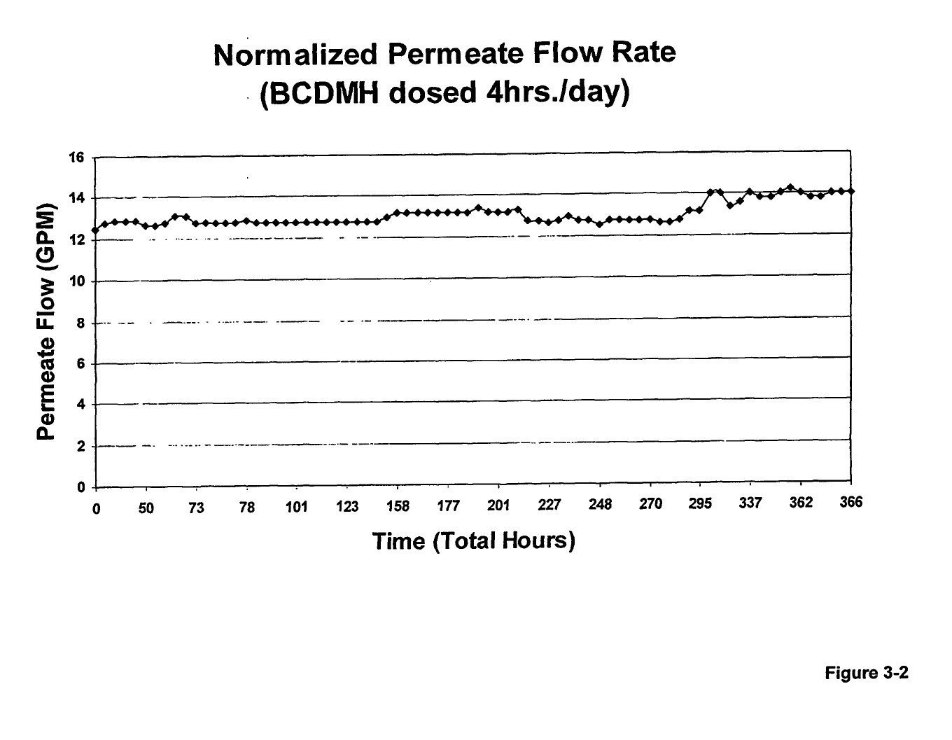

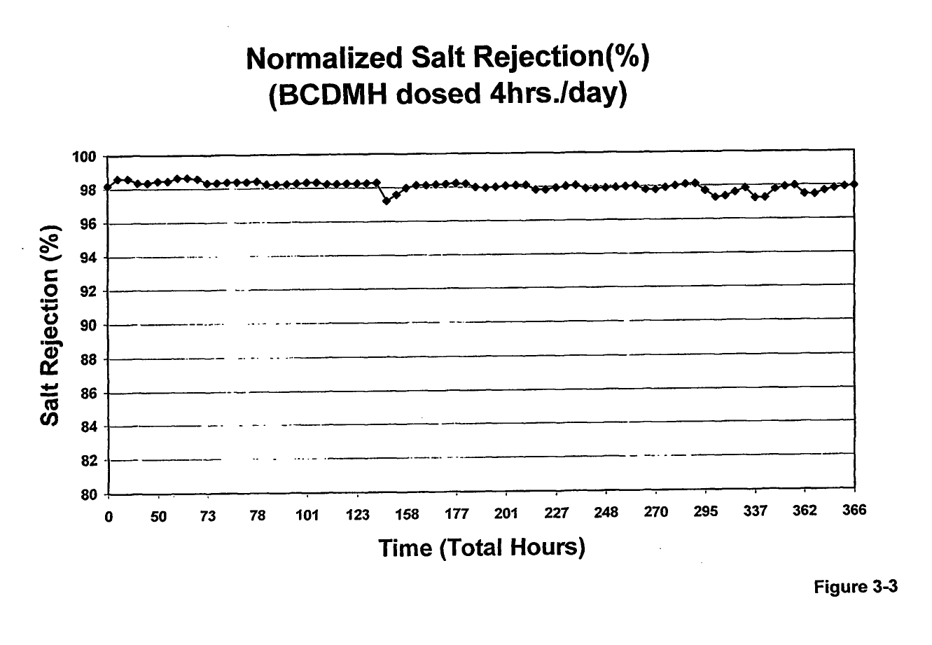

As can be seen when comparing FIGS. 2-2 and 2-3, permeate flux was much less affected by the BCDMH than with the Sodium Hypochlorite. Permeate flux rates dropped off immediately, even at lower dosage levels when feeding the Sodium Hypochlorite, while the BCDMH permeate flux rate decline was much more gradual.

### EXAMPLE 3

### Seawater RO Field Test

Conditions of operation- FIG. 3-1 provides an overall schematic of the field trial test system.

| | |
|---|---|
| Water Type: | Seawater (Atlantic Ocean) |
| Products Testing | Bromochlorodimethylhydantoin (BCDMH); |
| | BromiCide ® Gel from BioLab, Inc. |
| Recovery: | 43% |
| Feed Pressure: | 830 psig (57 barg) |
| Concentrate Pressure: | 790 psig (54 barg) |
| BCDMH dosing: | Intermittent |

Field Test at St. Croix, USVI

Concurrently with the lab tests, a more realistic study was carried out on commercially available membrane elements.

| | |
|---|---|
| Membrane type: | Thin film composite-(Koch TFC ® 2822SS |
| | Seawater Membranes) |
| Element size: | 8″ × 40″ |
| Parameters monitored: | Feed/Permeate/Concentrate flows |
| | Feed/Concentrate pressures |
| | Total halogen |
| | Feed/Permeate conductivity |
| | PH |
| Method used to measure | To monitor the amount of halogen, a DPD (N, |
| halogen: | N-diethyl-p-phenylenediamine) test kit was |
| | used. |

Findings/Conclusions:
FIG. 3-2 indicates that the normalized permeate flow rates slightly increased during the course of the field test. If membrane surfaces were damaged by the halogen, it would be expected to see normalized permeate flow rates rise significantly. Therefore it can be concluded that the treatment with BCDMH resulted in no significant membrane degradation.
As illustrated in FIG. 3-3, normalized salt rejection rates remained steady. This is also an indicator that no adverse affects were noticed when feeding the BCDMH. It would be expected to see a rise in salt passage and a decline in salt rejection if membrane surfaces were being damaged.

### EXAMPLE 4

### Seawater RO Pilot Plant Test

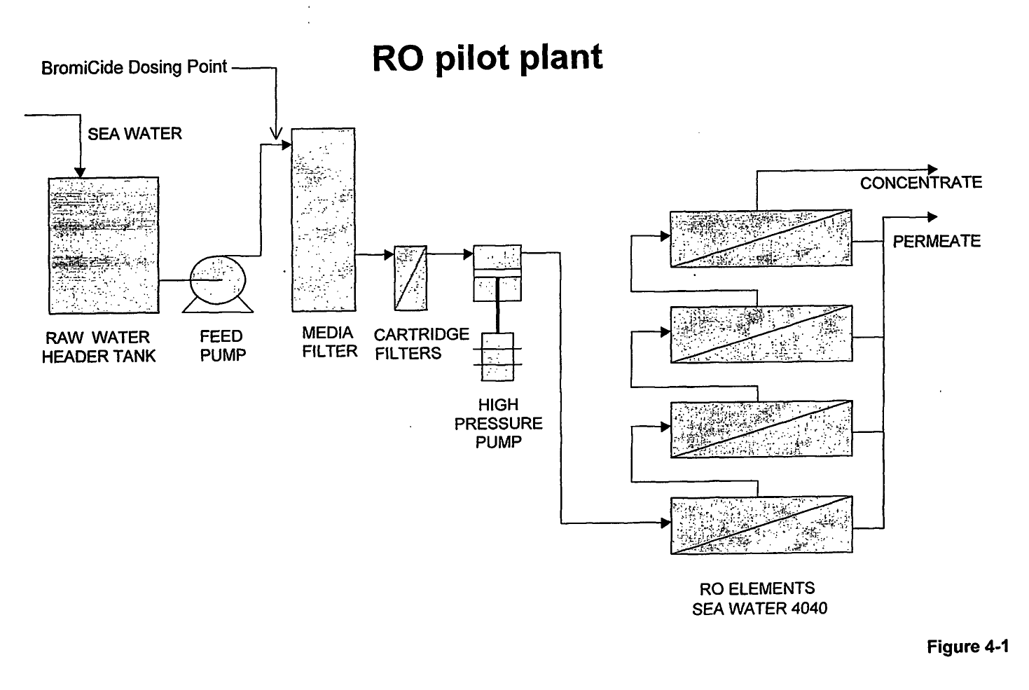

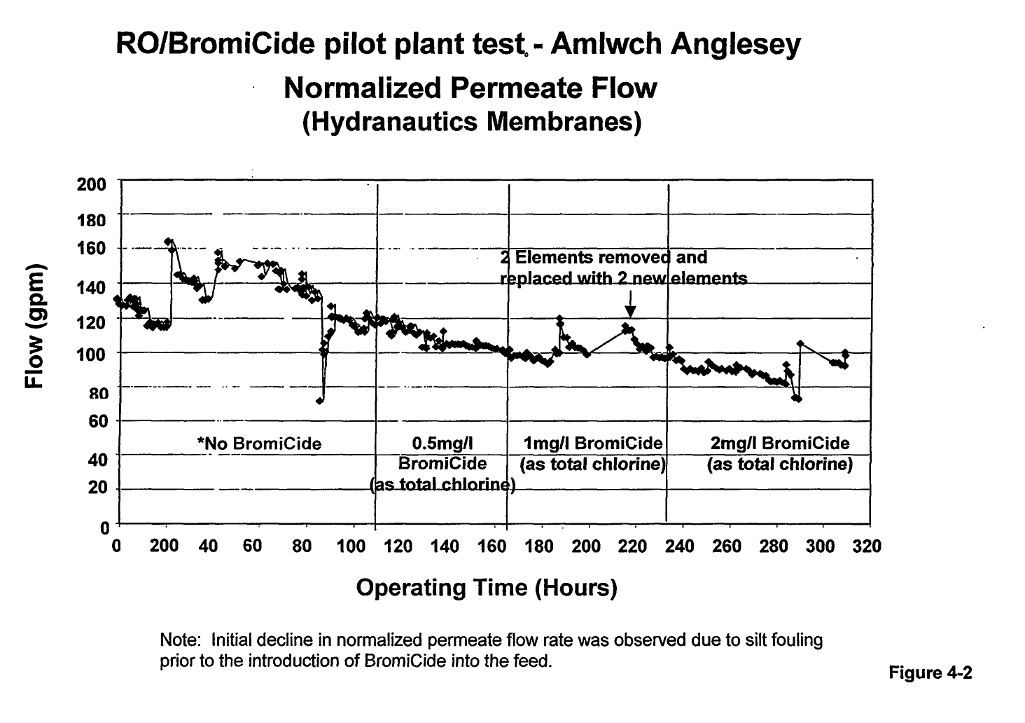

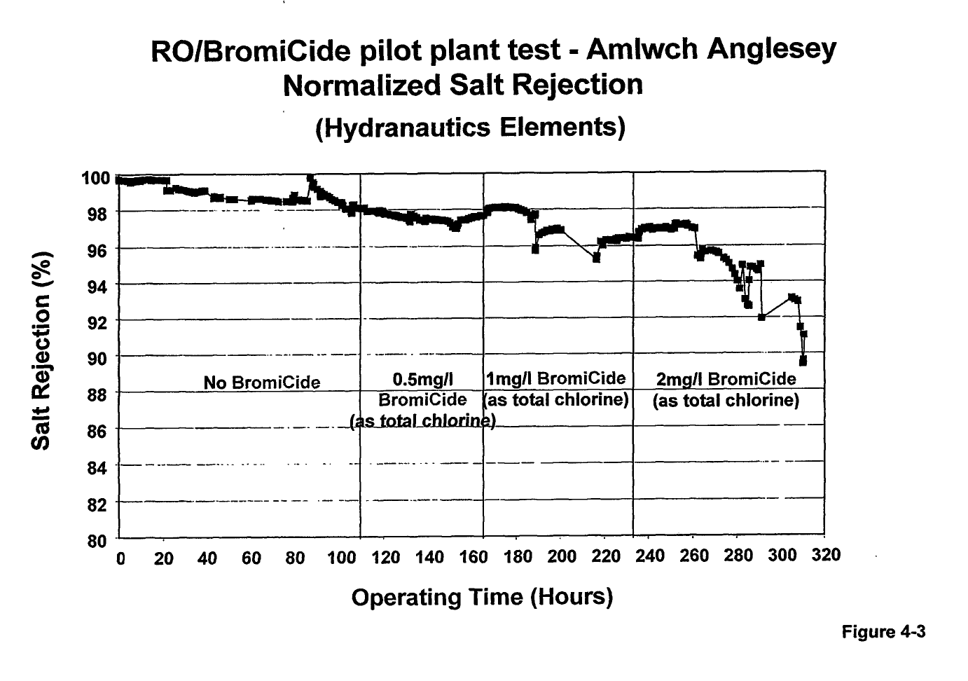

Pilot plant test was carried out at Amlwch, Anglesey in North Wales, UK. FIG. 4-1 provides an overall schematic of the field trial test system.

Conditions of operation

| | |
|---|---|
| Membrane Type 1: | Thin film composite |
| | (Hydranautics SWC2-4040) |
| Element Size: | 4″ × 40″ |
| Parameters monitored: | Feed/Permeate/Concentrate flows |
| | Feed/Concentrate pressures |
| | Total halogen |
| | Feed/Permeate Conductivity |
| | pH |
| Method used to measure | To monitor the amount of halogen, a DPD (N, |
| halogen: | N-diethyl-p-phenylenediamine) test kit was |
| | used. |
| Bacteria Monitoring: | Nutrient agar dip slides were used to monitor |
| | seawater and RO feed water bacteria levels. |
| Water type: | Seawater (Irish Sea) |
| Products Tested | Bromochlorodimethylhydantoin (BCDMH); |
| | BromiCide ® |
| | Gel from BioLab, Inc. |
| Recovery: | 25% |
| Feed pressure: | 65 barg (943 psig) |
| Concentrate pressure: | 60 barg (870 psig) |
| BCDMH dosing: | Continuous |

Findings/Conclusions:
FIGS. 4-2 and 4-3 provide illustrations that exposure of the Hydranautics membranes to BCDMH did not result in a reduction in normalized permeate and % salt rejection. Problems with initial silt fouling caused declines in both of these monitored parameters, before BCDMH was introduced into the system, and this trend continued at the same rate. This indicates that the BCDMH did not have a negative effect on these two characteristics.
Results of bacteria monitoring showed the raw seawater to be infected with bacteria, while the RO feedwater, dosed with BCDMH, was found to be clear. This clearly demonstrated BCDMH's ability to control biofouling in the RO pilot plant throughout the trial period.
   General Conclusions

As indicated in each of the four examples above, it is clearly shown that BCDMH is much more tolerated by polyamide surfaces than is the Sodium Hypochlorite.

Further variations and modifications will be apparent to those skilled in the art from the foregoing and are intended to be encompassed by the claims appended hereto.

TABLE I
Cross Sectional Areas of Fibers
Cross Sectional Areas of Differing Fibers

| Sample | Cross Sectional Area (μ² × 10³) | | |
|---|---|---|---|
| Number | BCDMH | Activated NaBr | Bleach |
| Blank | 1.19 | 1.19 | 1.19 |
| 1 | 1.23 | 1.12 | 1.11 |
| 2 | 1.19 | 1.14 | 1.15 |
| 3 | 1.09 | 1.11 | 1.22 |
| 4 | 1.09 | 1.10 | 1.21 |

We claim:

1. A process for treating a reverse osmosis membrane with a biocide to kill bacteria on or in the vicinity of said membrane comprising contacting said reverse osmosis membrane with an oxidizing halogen biocide in combined form which slowly releases said halogen in sufficient amount to disinfect said membrane and to kill said bacteria and thereby eliminate or prevent biofilm on said membrane.

2. The process according to claim 1 wherein the halogen biocide is a combination of an oxidizing biocide substance that contains a halogen in the +1 oxidation state and a nitrogen containing compound which contains at least one nitrogen atom in the imide or amide form, such that the halogen loosely binds with the nitrogen thereby forming combined halogen.

3. The process according to claim 1 wherein the oxidizing biocide is a halogen containing biocide that includes nitrogen in the imide or amide form.

4. The process according to claim 2 wherein the halogen is bromine.

5. The process according to claim 1 wherein the biocide is bromochlorodi-methylhydantoin.

6. A process for treating water with a reverse osmosis membrane for desalination of said water, comprising contacting said water upstream from said membrane with an oxidizing halogen biocide to kill bacteria on or in the vicinity of said membrane, wherein said biocide contains an oxidizing halogen in combined form which slowly releases said halogen in sufficient amount to disinfect said membrane and to kill said bacteria and thereby eliminate or prevent a biofilm on said membrane.

7. The process according to claim 6 wherein the halogen biocide is a combination of an oxidizing biocide substance that contains a halogen in the +1 oxidation state and a nitrogen containing compound that contains at least one nitrogen atom in the imide or amide form, such that the halogen loosely binds with the nitrogen thereby forming combined halogen.

8. The process according to claim 6 wherein the oxidizing biocide is a halogen containing biocide that includes nitrogen in the imide or amide form.

9. The process according to claim 6 wherein the halogen is bromine.

10. The process according to claim 6 wherein the biocide is bromochlorodi-methylhydantoin.

11. The process according to claim 6 further comprising providing said biocide in the form of a solid compact, dissolving said solid compact to form a concentrated solution of said biocide and feeding said concentrated solution of the biocide into the water.

12. The process according to claim 11 wherein the concentrated solution is fed into the water at such a rate so as to provide from 0.05 to 4 ppm total halogen at the point of contact with the membrane.

13. The process for treating water with a reverse osmosis membrane according to claim 6 wherein a suspension or solution containing said biocide is introduced into a stream of water at such a rate so as to provide from 0.05 to 4 ppm total halogen on said membrane to kill said bacteria and thereby eliminate or prevent a biofilm on said membrane.

14. The process according to claim 13 wherein the biocide is a halogen containing compound that includes nitrogen in the imide or amide form.

15. The process according to claim 14 wherein the halogen is bromine.

16. The process according to claim 13 wherein the biocide is bromochlorodi-methylhydantoin.

17. A process for treating a reverse osmosis membrane made of polyamide with a biocide to kill bacteria on said membrane comprising contacting said reverse osmosis membrane with a stream of water containing an oxidizing biocide that contains a halogen in combined form which slowly releases said halogen in sufficient amount to disinfect said membrane and to kill said bacteria and thereby eliminate or prevent a biofilm on said membrane.
